(19) United States
(12) Patent Application Publication
(10) Pub. No.: US 2006/0229670 A1
(43) Pub. Date: Oct. 12, 2006

(54) Method and a medical closure system for sealing a puncture

(76) Inventor: Brian L. Bates, Bloomington, IN (US)

Correspondence Address: BRINKS HOFER GILSON & LIONE/CHICAGO/COOK, PO BOX 10395, CHICAGO, IL, 60610, US

(21) Appl. No.: 11/396,377

(22) Filed: Mar. 30, 2006

Related U.S. Application Data
(60) Provisional application No. 60/667,251, filed on Apr. 1, 2005.

(51) Int. Cl. A61B 17/08

(52) U.S. Cl. 606/213

(57) ABSTRACT

The invention is generally directed to a method for sealing a puncture through a wall of a blood vessel or wall of a body cavity. The invention is also directed to a medical closure system including a closure member, an occlusive material or a composition of occlusive materials.

### RELATED APPLICATIONS

The present patent document claims the benefit of the filing date under 35 U.S.C. §119(e) of Provisional U.S. Patent Application Ser. No. 60/667,251, filed Apr. 1, 2005, which is hereby incorporated by reference.

### BACKGROUND OF INVENTION

1. Technical Field

This invention relates to a method and a system that facilitate closure and sealing of openings in tubular tissue structures or the wall of a body cavity. More specifically, the present invention is directed to a method and a system for closing a puncture in the wall of a tubular tissue structure, or in the wall of a body cavity using a medical closure system including a closure member, a reconstituted or naturally-derived collagenous material, such as an extracellular matrix (ECM) and a hemostatic material (i.e., occlusive material).

2. Background Information

The control of bleeding during and after surgery is important to the success of the procedure. The control of blood loss is of particular concern if the surgical procedure is performed directly upon or involves the patient's arteries and veins.

Typically, the insertion of a catheter creates a puncture through the vessel wall and upon removal, the catheter leaves a puncture opening through which blood may escape and leak into the surrounding tissues. Therefore, unless the puncture site is closed, clinical complications may result leading to increased hospital stays with the associated costs. To address this concern, medical personnel are required to provide constant and continuing care to a patient who has undergone a procedure involving an arterial or venous puncture to ensure that post-operative bleeding is controlled.

Surgical bleeding concerns can be exacerbated by the administration of a blood thinning agent, such as heparin, to the patient prior to a catheterization procedure. Since the control of bleeding in anti-coagulated patients is much more difficult to control, stemming blood flow in these patients can be troublesome. A common method of healing the puncture to the vessel is to maintain external pressure over the vessel until the puncture seals by natural clot formation processes. This method of puncture closure typically takes about thirty to ninety minutes, with the length of time usually being greater if the patient is hypertensive or anti-coagulated.

Furthermore, it should be appreciated that utilizing pressure, such as human hand pressure, to control bleeding suffers from several drawbacks regardless of whether the patient is hypertensive or anti-coagulated. In particular, when human hand pressure is utilized, it can be uncomfortable for the patient, can result in excessive restriction or interruption of blood flow, and can use costly professional time on the part of the hospital staff. Other pressure techniques, such as pressure bandages, sandbags, or clamps require the patient to remain motionless for an extended period of time and the patient must be closely monitored to ensure the effectiveness of these techniques.

Devices have been disclosed which plug or otherwise provide an obstruction in the area of the puncture (see, for example, U.S. Pat. Nos. 4,852,568 and 4,890,612) wherein a collagen plug is disposed in the blood vessel opening. When the plug is exposed to body fluids, it swells to block the wound in the vessel wall. A potential problem with plugs introduced into the vessel is that particles may break off and float downstream to a point where they may lodge in a smaller vessel, causing an infarct to occur. Another potential problem with collagen plugs is that there is the potential for the inadvertent insertion of the collagen plug into the lumen of the blood vessel which is hazardous to the patient. Collagen plugs also can act as a site for platelet aggregation, and, therefore, can cause intraluminal deposition of occlusive material creating the possibility of a thrombosis at the puncture sight. Other plug-like devices are disclosed, for example, in U.S. Pat. Nos. 5,342,393; 5,370,660; and 5,411,520.

Although efforts have been made to close puncture wounds using collagen plugs, and other means such as staples, clips, sutures, these efforts have been unsuccessful, largely due to the inability to locate the puncture wound in the vessel, such as femoral artery, and also because of the difficulty of controllably modifying the artery in the limited space provided.

Thus, a device and method to facilitate locating area adjacent to the puncture wound and closing of such wounds in the vasculature or in the wall of a body cavity, such as a heart chamber, or a body cavity of another organ of a patient would be extremely beneficial. A device having the ability to consistently, reliably, and quickly close the puncture wound eliminate the prolonged bleeding currently associated with such wounds, prevent disposing any occlusive material into the vessel or body cavity, and prevent introducing infectious organisms into the patient's circulatory system.

### SUMMARY OF INVENTION

In one embodiment, the invention is a method for sealing a puncture through a wall of a blood vessel or wall of a body cavity. The method includes deploying a closure member in a first compacted configuration through a delivery member into the blood vessel or body cavity though the puncture, wherein the closure member radially expands to assume a second expanded configuration following the deployment. The method also includes positioning the closure member on an inner surface of the blood vessel or body cavity at the puncture and delivering a reconstituted or naturally-derived collagenous material and a hemostatic material through the delivery member to an outer surface of the blood vessel or body cavity at the puncture. The closure member defines an area for delivering of the reconstituted or naturally-derived collagenous material and the hemostatic material at the puncture. The method may further include retracting the closure member through the delivery member. The reconstituted or naturally-derived collagenous material and the hemostatic material may be delivered via at least one sideport positioned on a distal end of the delivery member.

In another embodiment, the invention is a method for sealing a puncture through a wall of a blood vessel or wall of a body cavity. The method includes deploying a closure member in a first compacted configuration through a delivery member into the blood vessel or body cavity though the puncture, wherein the closure member radially expands to assume a second expanded configuration following the deployment. The method also includes positioning the closure member on an inner surface of the blood vessel or body cavity at the puncture and delivering a composition comprising a reconstituted or naturally-derived collagenous material and a hemostatic material through the delivery member to an outer surface of the blood vessel or body cavity at the puncture. The closure member defines an area for delivering of the reconstituted or naturally-derived collagenous material and the hemostatic material at the puncture.

In yet another embodiment, the invention is a medical closure system for sealing a puncture through a wall of a blood vessel or wall of a body cavity. The medical closure system includes a closure member which can be in a first collapsed configuration or in a second expanded configuration, a reconstituted or naturally-derived collagenous material, and a hemostatic material. The system may further comprise a delivery member, which may include at least one sideport positioned at a distal end of the delivery member. The reconstituted or naturally-derived collagenous material may include an ECM material, preferably in an injectable form. The hemostatic material may be selected from the group consisting of HEMOS, Fibrin adhesive material, Epsilon-Aminocapronic Acid, Chitosan, poly-N-acetylglucosamine, Microporous polysaccharide hemosphere, QR powder, and hemostatic lipid.

In yet another embodiment, the invention is a medical closure system for sealing a puncture through a wall of a blood vessel or in the wall of a body cavity. The medical closure system includes a closure member which can be in a first collapsed configuration or in a second expanded configuration, and a composition comprising a reconstituted or naturally-derived collagenous material and a hemostatic material. The system may further include a delivery member, which may include at least one sideport positioned at a distal end of the delivery member.

### BRIEF DESCRIPTION OF THE DRAWINGS

FIG. 1 is a schematic illustration of a delivery member and deployment of the medical closure system;

FIG. 2 is a schematic illustration of a delivery member and deployment of the medical closure system;

FIG. 3 is a schematic illustration of a delivery member and deployment of the medical closure system;

FIG. 4 is a schematic illustration of a delivery member and deployment of the medical closure system.

FIGS. 5A and 5B are illustrations of an exemplary closure member;

FIGS. 6A and 6B are illustrations of another closure member;

FIGS. 7A and 7B each depict a metal fabric suitable for use with the invention;

FIGS. 8A and 8B depict alternate closure member; and

FIG. 9 is a view of an embodiment of a closure member.

### DETAILED DESCRIPTION OF THE DRAWINGS AND THE PRESENTLY PREFERRED EMBODIMENTS

There is a need in the art for a method and device for sealing a wound or puncture in a vessel or organ wall.

The present method and closure system are especially useful for closing vascular and other puncture wounds that are difficult to access and/or locate. It is difficult to directly and accurately modify the wounded blood vessel in order to close such wounds. Additionally, there are pitfalls associated with directly modifying the blood vessel. For example, it may be difficult for a physician to correctly place an occlusive material. Incorrect placement of the occlusive material likely may result in inadequate closure; the puncture wound remaining open, perhaps without the clinician being aware. Additionally, incorrect placement of the occlusive material may cause permanent damage to the vessel, including tearing and additional puncture wounds. Further, if the occlusive material extends through the wound and into the blood flow, this material may increase the likelihood of thrombus formation or could introduce potentially toxic substances into the bloodstream. Of course, occlusive material inadvertently released into the bloodstream could lead to serious blood vessel blockage complications.

The present invention overcomes these pitfalls by providing a method and device for defining an area adjacent to the puncture and quickly sealing punctured tubular tissue structures, including arteries and veins, or punctured wall of a body cavity, such as a heart chamber, or a body cavity of another organ by delivering occlusive material to that area. Specifically, the method and device of the present invention employ a closure member and an occlusive material. It is the placement of the closure member at the puncture that defines a location for delivery of the occlusive material and provides information on the position of the closure member relative to the distal end of a delivery member. Preferably, the method and device of the present invention employ a reconstituted or naturally-derived collagenous material, such as submucosal tissue or another ECM-derived tissue; hemostatic material; and a closure member.

The naturally-derived collagenous material, such as submucosal tissue or other ECM-derived tissue, is capable of inducing tissue remodeling at the site of implantation by supporting the growth of connective tissue in vivo while containing the hemostatic material. It also has the added advantages of being tear-resistant so that occlusive material is not introduced into the patient's circulatory system. Also, naturally-derived collagenous material, such as submucosal tissue or another ECM-derived tissue, has the advantage of being resistant to infection, thereby reducing the chances that a procedure will result in systemic infection of the patient.

The hemostatic material, on the other hand, allows for controlling bleeding to restore hemostasis after the procedure.

Lastly, the closure member provides information on the exact location for delivering of the occlusive materials, such as ECM and hemostatic agent and it also prevents these occlusive materials from entering the vessel and patient's circulatory system, or an organ. The location is provided by knowing the position of the closure member relative to the distal end of a delivery device.

The method and device of the present invention can be used to seal an opening or a puncture in a tubular tissue structure, such as a blood vessel, or in the wall of a body cavity, that has been created intentionally or unintentionally during a surgical procedure, such as punctures which have been created during diagnostic and interventional vascular and peripheral catheterizations, or nonsurgically (e.g., during an accident). Punctures made intentionally include vascular punctures made in various types of vascular, endoscopic, or orthopaedic surgical procedures, or punctures made in any other type of surgical procedure, in coronary and in peripheral arteries and veins or in the wall of a body cavity. Such procedures include angiographic examination, angioplasty, laser angioplasty, valvuloplasty, atherectomy, stent deployment, rotablator treatment, aortic prosthesis implantation, intraortic balloon pump treatment, pacemaker implantation, any intracardiac procedure, electrophysiological procedures, interventional radiology, and various other diagnostic, prophylactic, and therapeutic procedures such as dialysis and procedures relating to percutaneous extracorporeal circulation.

In the discussions herein, a number of terms are used. In order to provide a clear and consistent understanding of the specification and claims, the following definitions are provided.

“Hemostatic material” refers to any material capable of restoring homeostasis in an injured, punctured or otherwise diseased vessel or body cavity. Examples of suitable hemostatic materials are described below.

“Occlusive material” refers to any material suitable for occluding or closing a puncture site in a wall of a blood vessel or body cavity. For example, occlusive material includes various types of naturally-derived collagenous material, such as ECM; hemostatic materials; or mixtures thereof. Various types of occlusive materials are described in more detail below.

“Bioburden” refers to the number of living microorganisms, reported in colony-forming units (CFU), found on and/or in a given amount of material. Illustrative microorganisms include bacteria, fungi, and their spores.

“Disinfection” refers to a reduction in the bioburden of a material.

“Sterile” refers to a condition wherein a material has a bioburden such that the probability of having one living microorganism (CFU) on and/or in a given section of the material is one in one-million or less.

“Purification” refers to the treatment of a material to remove one or more contaminants which occur with the material, for instance contaminants with which the material occurs in nature, and/or microorganisms or components thereof occurring on the material. Illustratively, the contaminants may be those known to cause toxicity, infectivity, pyrogenicity, irritation potential, reactivity, hemolytic activity, carcinogenicity and/or immunogenicity.

“Biocompatibility” refers to the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests assay as to a material's toxicity, infectivity, pyrogenicity, irritation potential, reactivity, hemolytic activity, carcinogenicity, and/or immunogenicity. A biocompatible structure or material when introduced into a majority of patients will not cause an adverse reaction or response. In addition, it is contemplated that biocompatibility can be effected by other contaminants such as prions, surfactants, oligonucleotides, and other biocompatibility effecting agents or contaminants.

“Contaminant” refers to an unwanted substance on, attached to, or within a material. This includes, but is not limited to: bioburden, endotoxins, processing agents such as antimicrobial agents, blood, blood components, viruses, DNA, RNA, spores, fragments of unwanted tissue layers, cellular debris, and mucosa.

“Catheter” refers to tube that is inserted into a blood vessel to access the vessel. Catheter includes catheter per se, inducer sheath and other suitable medical devices.

Method of Sealing a Puncture Site

The invention is a method of sealing a puncture though a wall of a blood vessel or a wall of a body cavity. The method includes deploying a closure member in a first compacted or collapsed configuration though a delivery member as described herein below. The closure member may be deployed into the blood vessel or body cavity though the puncture, wherein the closure member radially expands to assume a second expanded configuration following the deployment. The closure member may be positioned on an inner surface of the blood vessel or body cavity at the puncture to define an area for delivering of the reconstituted or naturally-derived collagenous material and the hemostatic material at the puncture. The method also includes delivering the occlusive material including ECM and hemostatic materials, or a composition comprising both at the puncture.

The remaining details of the method relate to the method of delivering the medical closure system of this invention as described below.

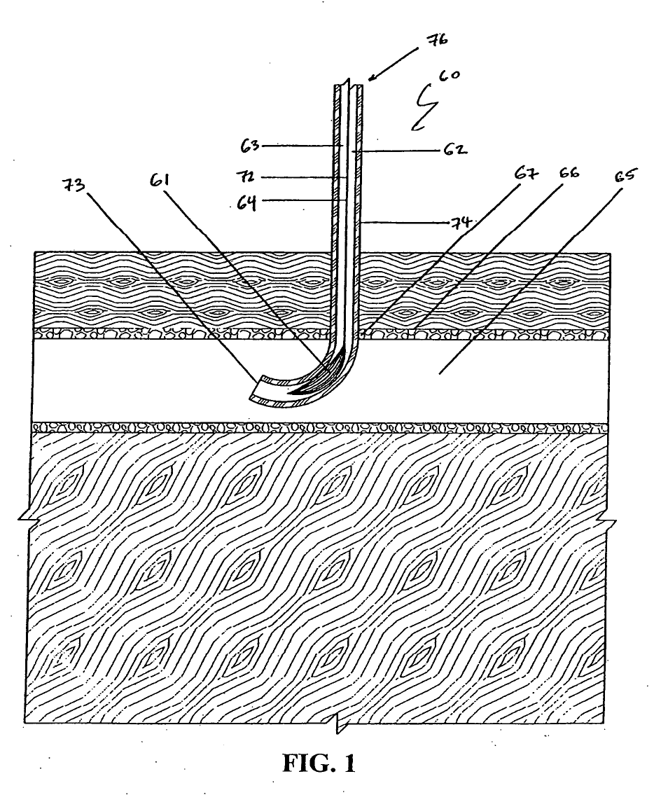

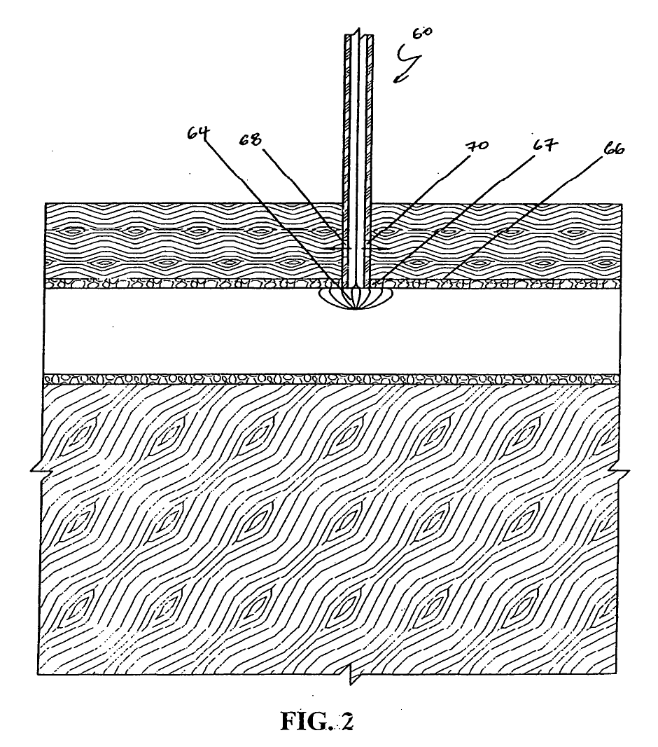

Referring to FIGS. 1-4, first, a basket portion 61 of the closure member 64, formed in a predetermined shape, and made in accordance with the process outlined below, can be collapsed and inserted into the lumen of a delivery member, as shown in FIG. 1. A delivery member 60 may take any suitable shape, but desirably comprises a catheter comprising a body 74 having a plurality of lumens 62 and 63 extending longitudinally therein. At least one of the lumens extending longitudinally through the catheter body 74 from its proximal end 76 to an exit port at its distal end 73, and at least one other lumen extends longitudinally through the catheter body to a closed distal portion of the catheter body. The lumen with an exit port may be adapted for delivering a closure member 64, comprising, for example a collapsible basket 61 and a shaft 72, into a lumen 65 of a vessel 66 and positioning the closure member 64 adjacent to an inner surface of a puncture site 67. As shown in FIG. 2, the catheter body may further include at least one sideport 68 positioned to deliver occlusive materials, including ECM and/or hemostatic materials, or a composition comprising both, to an area exterior to the catheter body and exterior to the vessel but in close proximity to a puncture site 67 in a wall of the vessel 66. Sideports are openings that are cut or otherwise formed in a catheter body 74 in a known manner. The sideports are preferably positioned at the far distal end of the catheter.

Two sideports 68 and 70 may be included on the catheter body 74. Multiple sideports may be provided (not shown) and are preferred. Sideports may be separated, or spaced, an appropriate distance along the body and the length of the catheter in proportion to the catheter's French size. Such configuration would be desired to increase distribution points for delivering of occlusive material. In a preferred embodiment, sideports may be adjacent to each other to allow for a quick and efficient delivery of the occlusive material.

An exemplary delivery device was previously described in U.S. Pub. No. 2005/0004554, disclosure of which is incorporated herein in its entirety.

In addition, a catheter used for delivering of the medical closure system of this invention may include a sheath.

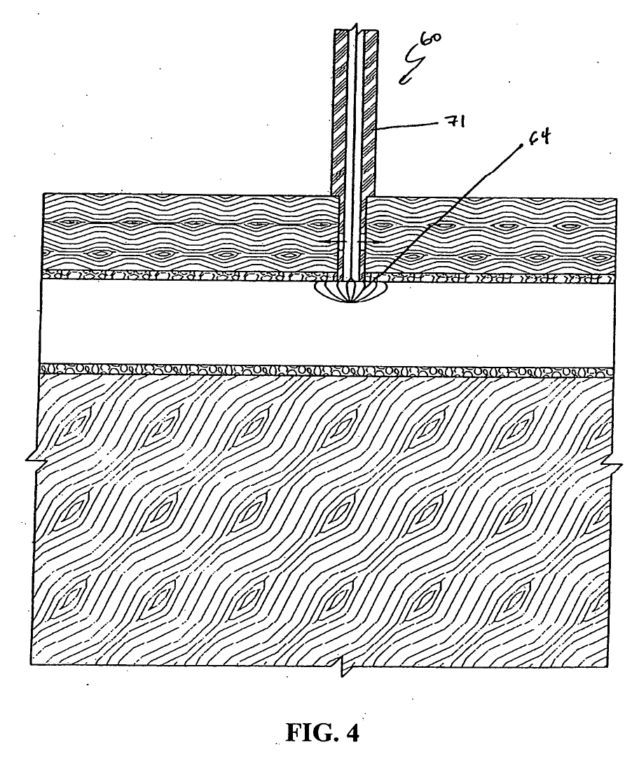

Also, as shown in FIG. 4, the catheter may include a coaxial member 71, such as a collar or an umbrella-like structure to preclude the catheter from going back into the vessel following deployment and positioning of the closure member 64 as described below. This coaxial member may be adjustable in size. Alternatively, the catheter may include a build-in profile into the catheter itself to prevent the catheter from entering the vessel following deployment and positioning of the closure member.

The closure member 64 may then be advanced through the lumen of a catheter to extend beyond the distal end of the catheter (not shown) for deployment in a tubular tissue structure, such as a blood vessel 65 or other structure and for further delivery of the occlusive material. Upon exit, the closure member elastically expands to substantially recover its thermally set, “remembered” shape from the heat treatment process and assume its expanded configuration.

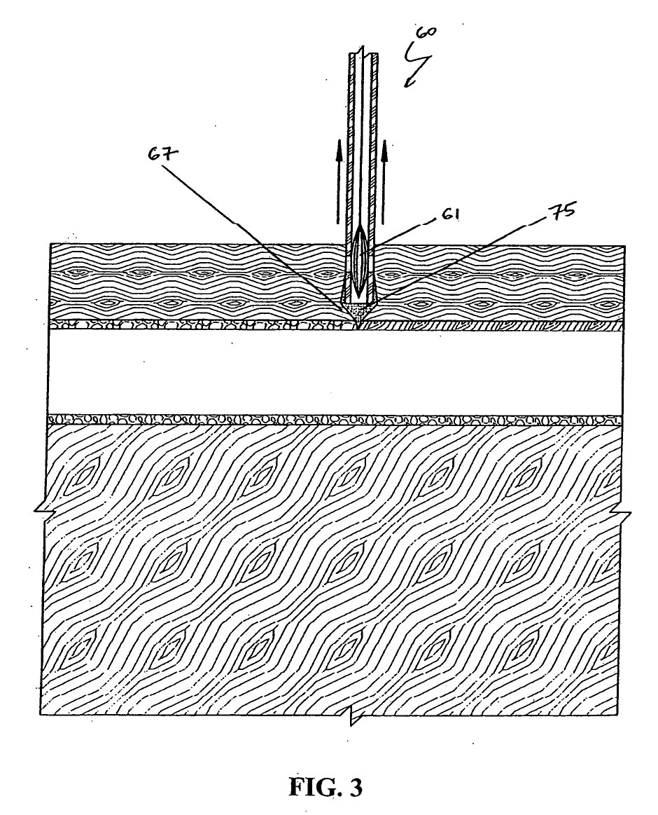

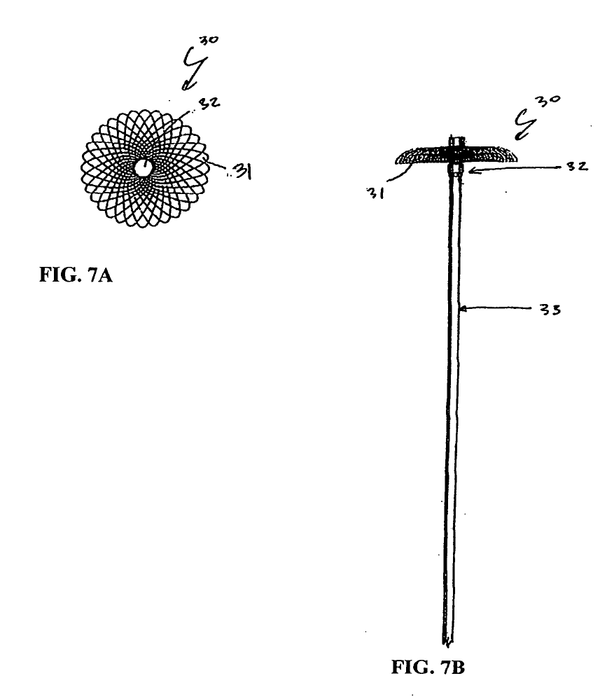

Once the device is deployed out the distal end 73 of the catheter 60, the closure member 64 may be retained by the delivery device as shown in FIG. 2. By keeping the closure member attached to the delivery means, the operator may still retract the closure member for repositioning if it is determined that the device is not properly positioned in the first attempt. The proper positioning may be confirmed by retracting the catheter back and confirming that no blood flow occurs through the catheter 60. The operator may further confirm the proper positioning by feeling for resistance when the closure member is retracted back to engage the vessel wall. Also, by keeping the closure member attached to the delivery means, the operator may identify the exact location for precise delivery of the occlusive material (arrows) at the puncture site 67 in a wall 66 of a tubular tissue structure, such as a blood vessel 65. This threaded attachment may further allow the operator to control the manner in which the closure member is deployed out of the distal end 73 of the catheter 60. When the closure member 64 exits the catheter 60 it will tend to resiliently return to a preferred expanded shape which is set when the fabric is heat treated. When the device springs back into this shape, it may tend to act against the distal end of the catheter, effectively urging itself forward beyond the end of the catheter. This spring action could conceivably result in improper positioning of the device if the location of the device within a vessel is critical, such as where it is being positioned in a puncture site 67 of a blood vessel 65. A threaded clamp 32 shown in FIG. 7 may enable the operator to maintain a hold on the closure member during deployment, the spring action of the closure member may be controlled and the operator may control the deployment to ensure proper positioning. The threaded attachment may also allow the operator to collapse and permanently remove the closure member, as shown in FIG. 3, after delivery of the occlusive material 75.

As shown in FIG. 2, once the proper position of the closure member in the puncture site 67 is confirmed, the closure member 64 may be retracted by retracting the catheter so that the “flat” basket portion 61 of the closure member 64 engages the wall of the vessel covering the puncture site 67 from the inside of the vessel. By retracting the catheter the sideports 68 and 70 of the catheter are placed outside the vessel 65 in close proximity to the puncture site 67. The basket portion may preferably be sized so that it will frictionally engage the puncture site 67. Such positioning of the closure member allows for complete closure of the puncture site 67.

Once the sideports 68 and 70 of the catheter are placed outside the vessel 65 in close proximity to the puncture site 67 as described above, the occlusive materials including ECM and hemostatic materials, or compositions comprising both, may be injected through at least one sideport 68 or 70 of the catheter 60. The occlusive material may be released through the sideports 68 and 70 in a non-directional manner. Alternatively, the occlusive material may be directed to puncture site 67 in a wall of the vessel by a structure, such as a sheath (not shown). In addition, a directional placement of the occlusive material at the puncture site 67 may be achieved by angling the sideports at about 45 degrees.

As shown in FIG. 3, the occlusive material 75 may be placed on the outside of the vessel's puncture site 67. Preferred injectable ECM material polymerizes once delivered to the puncture site to form a collagenous support matrix to contain the hemostatic material. As previously mentioned, prior deployment of the closure member 64 at the puncture site 67 allows for precise placement of the sideports 68 and 70 and delivery of the occlusive material on the outside of the vessel 65. Also, prior deployment of the closure member 64 of the closure system of this invention prevents the occlusive material from entering the lumen of the vessel 65 through the puncture site 67.

Optionally, external or mechanical compression may be applied at the site for the recommended period of time or until the physician feels it is no longer necessary.

Following the delivery of the occlusive material 75 and positioning it over the puncture site 67, the closure member may be collapsed and retracted back through the distal end of the catheter. The catheter with the collapsible closure member 64 may then be withdrawn as shown by arrows in FIG. 3, leaving the occlusive material behind. The ECM functions to provide a structure and hemostatic material restores hemostasis to the vessel.

Other methods of placing the closure member at the puncture site may also be used. For example, the closure member may be placed at the puncture site using method of delivering a solid ECM material, such as SIS previously described in U.S. Pub. No. 2003/0051735, contents of which are incorporated herein.

In yet another embodiment, the delivery device may comprise an inducer sheath comprising at least one sideport and a small catheter also comprising sideports. Delivery devices comprising inducer sheath are known in the art. See for examples, U.S. Pat. No. 5,380,304. Examples of such devices used in renal, biliary, vascular, or other systems of a body were previously described in U.S. Pub. No. 2005/0043756, disclosure of which is incorporated in its entirety.

The delivery of the closure system of this invention may also occur via a rapid exchange delivery catheter. The rapid exchange delivery catheters and methods of using the rapid exchange delivery catheters were previously described in U.S. Pat. Nos. 4,762,129; 5,690,643; 5,814,061; 6,371,961; and Provisional Pat. Application, entitled “A Rapid Exchange Balloon Catheter and a Method for Making the Same,” Attorney reference LHa/129969.

Medical Device

The invention is also a medical device for closing of a puncture through a wall of a blood vessel or a wall of a body cavity, i.e., a medical closure system, comprising an occlusive material; and a closure member and is further described in the non-limiting disclosure set forth below.

1. Occlusive Material

The occlusive material may include a naturally-derived collagenous material, such as ECM material; at least one hemostatic material; and/or a composition comprising ECM material and at least one hemostatic material.

ECM Material

It is advantageous to use a remodelable material for the occlusive materials of the present invention, and particular advantage may be provided by including a remodelable collagenous material. Such remodelable collagenous materials can be provided, for example, by collagenous materials isolated from suitable tissue source from a warm-blooded vertebrate, and especially a mammal. Such isolated collagenous materials may be processed so as to have remodelable properties and promote cellular invasion and tissue infiltration. Remodelable materials may be used in this context to promote cellular growth at the puncture, while containing others materials, such as hemostatic materials, as described in detail herein.

Reconstituted or naturally-derived collagenous materials may be used as occlusive materials in the present invention. Such materials that are at least bioresorbable will provide advantage in the present invention, with materials that are bioremodelable and promote cellular invasion and ingrowth providing particular advantage.

Suitable bioremodelable materials may be provided by collagenous extracellular matrix materials (ECMs) possessing biotropic properties, including in certain forms angiogenic collagenous ECMs. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression.

Submucosa or other ECM materials may be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after delivery of the material in the patient. For example, as described in more detail below, a suitable hemostatic material may be applied to the ECM material.

Submucosa or other ECM tissue used in the invention is preferably highly purified, for example, as described in U.S. Pat. No. 6,206,931. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

Preferred type of submucosa for use in this invention is derived from the intestines, more preferably the small intestine, of a warm blooded vertebrate; i.e., small intestine submucosa (SIS). SIS is commercially available from Cook Biotech, West Lafayette, Ind.

Preferred intestine submucosal tissue typically includes the tunica submucosa delaminated from both the tunica muscularis and at least the luminal portions of the tunica mucosa. In one example the submucosal tissue includes the tunica submucosa and basilar portions of the tunica mucosa including the lamina muscularis mucosa and the stratum compactum. The preparation of intestinal submucosa is described in U.S. Pat. No. 4,902,508, and the preparation of tela submucosa is described in U.S. Pat. No. 6,206,931, both of which are incorporated herein by reference. The preparation of submucosa is also described in U.S. Pat. No. 5,733,337 and in 17 Nature Biotechnology 1083 (Nov. 1999); and WIPO Publication WO 98/22158, which is the published application of PCT/US97/14855. Also, a method for obtaining a highly pure, delaminated submucosa collagen matrix in a substantially sterile state was previously described in U.S. Pub. No. 2004 0180042 A1, disclosure of which is incorporated by reference.

In short, the stripping of the submucosa source is preferably carried out by utilizing a disinfected or sterile casing machine, to produce a submucosa which is substantially sterile and which has been minimally processed. A suitable casing machine is the Model 3-U-400 Stridhs Universal Machine for Hog Casing, commercially available from the AB Stridhs Maskiner, Gotoborg, Sweden. As a result of this process, the measured bioburden levels may be minimal or substantially zero. Other means for delaminating the submucosa source can be employed, including, for example, delaminating by hand.

In this method, a segment of vertebrate intestine, preferably harvested from porcine, ovine or bovine species, may first be subjected to gentle abrasion using a longitudinal wiping motion to remove both the outer layers, identified as the tunica serosa and the tunica muscularis, and the innermost layer, i.e., the luminal portions of the tunica mucosa. The submucosal tissue is rinsed with water or saline, optionally sterilized, and can be stored in a hydrated or dehydrated state. Delamination of the tunica submucosa from both the tunica muscularis and at least the luminal portions of the tunica mucosa and rinsing of the submucosa provide an acellular matrix designated as submucosal tissue. The use and manipulation of such submucosal tissue constructs for inducing growth of endogenous connective tissues is described and claimed in U.S. Pat. No. 5,281,422, the disclosure of which is incorporated herein by reference.

Following delamination, submucosa may be sterilized using any conventional sterilization technique including propylene oxide or ethylene oxide treatment and gas plasma sterilization. Sterilization techniques which do not adversely affect the mechanical strength, structure, and biotropic properties of the purified submucosa are preferred. Preferred sterilization techniques also include exposing the graft to ethylene oxide treatment or gas plasma sterilization. Typically, the purified submucosa is subjected to two or more sterilization processes. After the purified submucosa is sterilized, for example by chemical treatment, the matrix structure may be wrapped in a plastic or foil wrap and sterilized again using electron beam or gamma irradiation sterilization techniques.

Purified collagen-based materials used in the present invention may be processed in a number of ways, to provide collagenous materials useful both in vitro and in vivo.

For example, the ECM material for use in the present invention may be processed to provide preferred injectable compositions, including fluidized, comminuted, liquefied, suspended, and gel-like compositions, for instance using techniques for preparing a fluidized SIS described in U.S. Pat. Nos. 5,275,826 and 5,516,533, which are incorporated herein in their entirety. Injectable forms of the ECM material are preferred forms of ECM material for use in accordance with this invention.

In addition to injectable forms of ECM, the ECM material may take many other shapes and forms, such as coiled; helical; spring-like; randomized; branched; sheet-like; tubular; spherical; fragmented; powdered; ground; sheared; sponge-like; foam-like; and solid material shape.

With regard to injectable forms, solutions or suspensions of the ECM material may be prepared by comminuting and/or digesting the ECM material with a protease (e.g. trypsin or pepsin), for a period of time sufficient to solubilize the tissue and form substantially homogeneous solution. Interestingly, fluidizing ECM by comminuting or enzymatic degradation does not result in any appreciable loss of biotropic activities, as shown in U.S. Pat. No. 5,275,826.

The ECM starting material may be desirably comminuted by tearing, cutting, grinding, shearing or the like. Grinding the ECM material in a frozen or freeze-dried state is advantageous, although good results may be obtained as well by subjecting a suspension of pieces of the ECM material to treatment in a high speed blender and dewatering, if necessary, by centrifuging and decanting excess waste. The comminuted ECM material may be dried, for example freeze dried, to form a powder. Thereafter, if desired, the powder may be hydrated, that is, combined with water or buffered saline and optionally other pharmaceutically acceptable excipients, to form a fluid tissue graft composition, e.g., having a viscosity of about 2 to about 300,000 cps at 25EC. The higher viscosity compositions may have a gel or paste consistency. Preferred ECM material for use in accordance with present invention may be of injectable consistency.

In one illustrative preparation, the ECM material may be reduced to small pieces (e.g. by cutting) which are charged to a flat bottom stainless steel container. Liquid nitrogen may be introduced into the container to freeze the specimens, which may then be comminuted while in the frozen state to form a coarse ECM powder. Such processing can be carried out, for example, with a manual arbor press with a cylindrical brass ingot placed on top of the frozen specimens. The ingot serves as an interface between the specimens and the arbor of the press. Liquid nitrogen can be added periodically to the ECM material to keep it frozen.

Other methods for comminuting ECM material may be utilized to produce ECM powder usable in accordance with the present invention. For example, ECM material may be freeze-dried and then ground using a manual arbor press or other grinding means. Alternatively, ECM material may be processed in a high shear blender to produce, upon dewatering and drying, ECM powder.

Further grinding of the ECM powder using a prechilled mortar and pestle can be used to produce a consistent, more finely divided product. Again, liquid nitrogen is used as needed to maintain solid frozen particles during final grinding. The powder can be easily hydrated using, for example, buffered saline to produce an injectable ECM material for use in this invention at the desired viscosity.

To prepare another preferred injectable material, ECM powder may be sifted through a wire mesh, collected, and subjected to proteolytic digestion to form a substantially homogeneous solution. For example, the powder may be digested with 1 mg/ml of pepsin (Sigma Chemical Co., St. Louis, Mo.) and 0.1 M acetic acid, adjusted to pH 2.5 with HCl, over a 48 hour period at room temperature. After this treatment, the reaction medium can be neutralized with sodium hydroxide to inactivate the peptic activity. The solubilized ECM material may then be concentrated by salt precipitation of the solution and separated for further purification and/or freeze drying to form a protease-solubilized ECM material in powder shape.

The ECM material may be used as a heterograft for tissues, for example, vessels, in need of repair or augmentation most typically to correct trauma, including disease-induced tissue defects. The ECM material may also be used advantageously as a component of a medical closure system of this invention, either by itself, or in combination with a hemostatic material. The ECM material in combination with hemostatic material may be specifically used in tissue replacement, augmentation, and/or repair. These compositions can be used to induce regrowth of natural tissue in an area of an existent defect, such as puncture. By injecting or placing an effective amount of an ECM together with a hemostatic material into the locale of a tissue trauma or defect, including a puncture in a wall of a blood vessel or body cavity, one may readily take advantage of the biotropic and structural properties of the ECM in addition to hemostatic properties or blood coagulating properties of hemostatic material, as discussed previously.

Hemostatic Materials

Hemostatic materials that may be used with the ECM material according to this invention include, but are not limited to, HEMOS, Fibrin Adhesive Material (Tissucol®), Epsilon-Aminocaproic Acid (EACA), Chitosan, poly-N-acetylglucosamine (p-GIcNAc), Microporous Polysaccharide Hemosphere (MPH), QR powder, hemostatic lipids, and other suitable hemostatic materials, or mixtures thereof. Another hemostatic material, namely platelet aggregating material from equine arterial tissue, has been previously described in U.S. Pat. No. 4,374,830, disclosure of which is incorporated by reference.

Other suitable hemostatic materials known to those skilled in the art may also be used in accordance with this invention.

A hemostatic material may include, for example, HEMOS, chemical structure of which is shown in Formula 1 below.

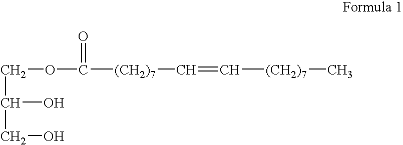

HEMOS is a monoglyceride that is obtained by esterifying glycerol with oleic acid from olive oil. HEMOS is widely used in the food industry as emulsifier and in pharmaceuticals as a drug carrier. HEMOS is characterized in that it is able to stop bleeding when applied to a hemorrhaging surface. HEMOS, when formulated with about 5% water content and epinephrine, exists as a liquid that can be poured, pumped, sprayed, mixed or otherwise applied to wound sites. Upon contacting blood, HEMOS absorbs fluid to form a wax-like structured “cubic” phase. The oil-like consistency of HEMOS allows for mixing of this hemotactic material with the ECM material. Preferably, HEMOS is mixed with ECM material in a fluidized or gel-like form, prepared as described above. The ECM material will provide the necessary structural component of this composition, while HEMOS allows to control bleeding and to restore hemostasis.

Manufacturing of HEMOS may involve two simple steps, as follows:

1. HEMOS, which is a white waxy solid in the pure state, may be prepared as a liquid by adding water for a final content of about 5%. Vasoactive, antimicrobial, and other small compounds may be added with the water. Mixtures may be sterilized by elevating the temperature.

2. The liquid HEMOS may be packaged as a liquid or composed with sponges, matrix, etc. as required, for example with the ECM material as described herein. A terminal sterilization with heat may be performed.

For use in this invention, HEMOS may be composed with an ECM material to form a composition comprising the ECM material and HEMOS. Such composition is prepared for delivery to seal a puncture site in a wall of a blood vessel, as part of the closure system.

Another example of hemostatic material includes Chitosan. Chitosan is a biodegradable, nontoxic, complex carbohydrate of chitin. “Chitin” is a polysaccharide that forms the ecoskeletons of insects and crustaceans. Chitosan may be derived from chitin by deacetylation (i.e. removal of the acetic acid radical CH₃CO⁻. Chitosan has been found to offer excellent hemostatic benefits (i.e. assist in blood clot formation). It is believed clotting is assisted by the ionic interaction between the positively charged chitosan polymer and the negatively charged red blood cell membrane. An advantage of this is that such clotting mechanism operates independently of the normal blood coagulation cascade which results in fibrin formation. Thus, chitosan can advantageously be used in conjunction with blood treated with heparin (which inhibits fibrin formation). In addition, chitosan is biodegradable: with the advantage that it is eventually re absorbed back into the body as a sugar.

Yet another example of hemostatic material includes Poly-N-acetylglucosamine (p-GIcNAc). P-GLcNAc may be derived from single-cell algae found in the ocean. It stimulates platelet aggregation and activation, which leads to the secretion of a substance known as tromboxane, which adds additional stimulus to enhance the local vasoconstriction of blood vessels in the vicinity of the wound. According to literature from Marine Polymer Technologies the data show that the hemostatic mechanism of poly-N-acetylglucosamine material acts as a catalytic surface that accelerates the normal clotting process resulting in the rapid control of bleeding. A distinct advantage of the p-GIcNAc is that is fully biodegradable and can be left in place on a bleeding surface to provide continued hemostasis after wound closure.

Yet another example of hemostatic material includes a wound-dressing agent utilizing Microporous Polysaccharide Hemosphere (MPH) Technology (Medafor, Inc.) that may be naturally synthesized from potato starch. When applied directly with pressure to an actively bleeding wound the particles may act to accelerate the natural blood clotting by concentrating blood solids, such as platelets and red blood cells, and other blood proteins such as albumin, thrombin and fibrinogen, to form a gel around the particles. The controlled porosity of the particle excludes platelets, red blood cells and serum proteins that are larger than 25,00 Dalton in size. The larger particles are then concentrated on the surface of the MPH particles.

This exclusion property of the MPH material creates a high concentration of platelets, thrombin, fibrinogen and other proteins on the particle surface, producing a gelling action. The gelled, compacted cells, thrombin and fibrinogen accelerate the normal clotting process. This gelling process has been shown to initiate within seconds.

Another example of hemostatic material include QR powder manufactured by Biolife, LLC. of Sarasota, Fla. The material is composed of a non-toxic mixture of a hydrophilic polymer and a potassium salt along with a bovine-based thrombin-based material.

A hemostatic material may also include, for example, fibrin tissue adhesive Tissucol®. Tissucol® used in patients with abnormal hemostatic function was previously found to serve the triple purpose of tissue sealing, hemostasis, and promotion of wound healing (Wepner et al., J Oral Maxillofac Surg. 1982 September;40(9):555-8; Matras, J Oral Maxillofac Surg. 1982 October;40(10):617-22; Matras, J Oral Maxillofac Surg. 1985 August;43(8):605-11; Baldin et al., G Stomatol Ortognatodonzia. 1985 April-June;4(2):69-75; Baudo et al., Haemostasis. 1985;15(6):402-4; Palattella et al., Dent Cadmos. 1985 Apr. 30;53(7):65-8, 71-3; Pini-Prato et al., Int J Periodontics Restorative Dent. 1985;5(3):32-41).

A hemostatic material may also include, for example a hemostatic lipid.

It is to be understood that the hemostatic materials may be in the form of a solid, gel or liquid. For example, when HEMOS is used, it may be used in the form of a solid, gel or liquid.

It is to be understood, however, that although the most preferred aspects of the present invention use HEMOS, the present invention is not so limited. Rather, any suitable hemostatic or blood clotting agent, including, but not limited to any form of HEMOS, may be used. In addition, other hemostatic materials such as fibrin and fibrinogen, may also be used instead of, or in addition to, the various presently contemplated hemostatic agents.

Compositions

A composition comprising both, the ECM material and the hemostatic material, may be prepared as described below.

Different forms of composition may be prepared. For example, the composition may be prepared in an injectable form. The injectable form may be prepared by mixing a comminuted ECM material with a hemostatic material to form a uniform composition comprising both, ECM and hemostatic material. Preferably, the ECM material may be pre-mixed with hemostatic material during the process of manufacturing the ECM, which was described above.

Also, a composition may be prepared in a sheet form, for example, by casting the composition described above and evaporating the solvent.

In one example, the hemostatic material may be added to the ECM after preparation of the ECM. For example, the ECM material in a solid form may be impregnated with the hemostatic material to provide the final composition. The term “impregnation” means providing for the presence of one or more components inside the ECM structure, in particular in the holes, such as interstices or pores of the ECM structure. Preferably, at least a substantial portion of the holes are open holes prior to treatment with hemostatic material. More preferably at least the majority of the total hole volume is provided by open holes. Open holes extend from one surface of the ECM material to another. Preferably at least a portion of the holes are filled with hemostatic material. The impregnation may partially or fully fill the holes of the ECM material. Preferably the impregnation is provided as a layer at least partially covering the inner surface of the holes, while maintaining a sufficient openness (porosity) to allow infiltration of cells or precursors thereof into the ECM material. By “infiltration” is meant cellular invasion upon delivery at the puncture. The process of cell or tissue infiltration may involve the invasion of inflammatory cells, fibroblasts, and other epithelial and mesenchymal cells from the surrounding tissue. The ECM may be impregnated with the hemostatic material that is in a fluid form, gel form or in a solid form, such as powder.

The hemostatic material may also be added to the ECM, for example by coating, lining, soaking, dipping, spraying, painting, and/or otherwise applying the hemostatic material to the ECM. Coating, dipping and spraying are conventional methods for impregnating the solution although dipping is preferred. For example, the ECM may be dipped into a bath containing the hemostatic material. The ECM material impregnated with the hemostatic material is then removed from the bath and allowed to dry. During the drying step, the solvent evaporates leaving the hemostaic material on the ECM.

The hemostatic material may also be incorporated into the ECM by binding it through photo-linking or other available means to the ECM material. Photo-linking, photo-activation, photo-polymerization, photo-crosslinking or photo-coupling refers to a process that is activated by light. A photo-activated step can be used to link the hemostatic material to the ECM material. The photo-activation step may require the presence of a photoinitiator, examples of which include acetophenones, benzophenones, hydroxipropiophenones, thioxanthones, diphenyl ketones, benzoin and benzoin alkyl ethers, halogen substituted alkylaryl ketones, or quinone and anthraquinone derivatives. The methods of photo-linking are known in the art.

The hemostatic material may also be immobilized on the ECM material by allowing interaction between the ECM material and the hemostatic material under conditions where a stable covalent or non-covalent linkage forms, e.g., by photo-crosslinking the hemostatic material if it and the surface comprise photo-activatable groups. “Stable” in this context refers to a linkage that is not disrupted during use of the fluidized ECM in a subsequent procedure, e.g., under washing or binding conditions. After immobilization, the surface can then be soaked, for example, in an aqueous buffer to remove non-covalently attached hemostatic material and excess cross-linking components and/or reagents.

The composition may be formed prior to delivering it to a puncture site in a wall of a blood vessel or body cavity. For example, the composition may be prepared preferably within about 3 hours, more preferably about 2 hours, and most preferably about 1 to about 0.5 hour of delivering it to the puncture location. Other time periods are also contemplated. For example, the composition may be prepared in advance (days, weeks, months) as part of a kit that also includes a closure member and may include a delivery device. In this instance, for example a fluidized ECM material may be pre-mixed with hemostatic material during the process of manufacturing the ECM material, which was described above, to form a composition comprising ECM and hemostatic material.

Alternatively, a composition may result from delivering the ECM material and the hemostatic material separately at the puncture in a wall of a blood vessel or body cavity during the medical procedure. Preferably, the ECM material may be delivered before the hemostatic material is delivered.

2. Closure Member

To provide a precise location for delivery of the occlusive material(s) at the puncture and to prevent the occlusive material from entering the vessel and patient's circulatory system, or an organ upon the delivery of the occlusive material, the medical closure system of this invention also includes a closure member. The closure member may comprise any suitable expandable medical device, and especially a medical device such as, for example, a collapsible basket, an expandable diaphragm, or a Malecot assembly. Preferably, however, the closure member comprises a collapsible basket.

Baskets are known in the retrieval art and are commonly used to remove an object, such as a stone or other undesirable object, from a body cavity. Examples of baskets were previously described in U.S. Pat. No. 5,725,552, disclosure of which is incorporated herein in its entirety. U.S. Pub. No. 2003/0171772 A1, disclosure of which is incorporated herein in its entirety, specifically discloses examples of collapsible baskets. Baskets have not been used, however, to provide a precise location for delivery of the occlusive material(s) to the outside of the wall of the vessel at a puncture and to prevent the occlusive material from entering the vessel and patient's circulatory system upon the delivery of the occlusive material.

A basket for intravascular occlusion according to this invention may be made from tubular mesh, which may be compressed for delivery through catheter (collapsed configuration) but which, on delivery, expands into a “flat” or “disc-like” shape (expanded configuration) appropriate for sealing a puncture site. Accordingly, a preferred basket for use in this invention may have an expanded configuration and a collapsed configuration. Preferably, the diameter of the basket in the expanded configuration is about 5 mm to about 100 mm; more preferably the diameter is about 5 mm to about 50 mm.

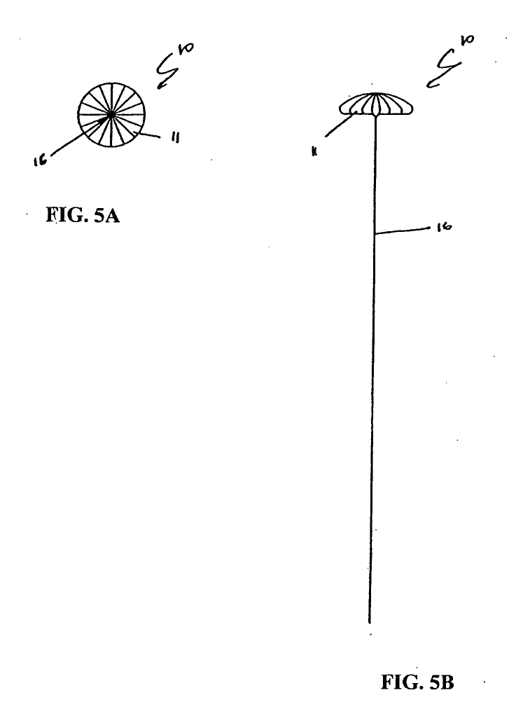

As illustrated in FIGS. 5A and 5B, the closure member 10, comprising a basket, which when in its unconstrained state, comprises a “flat” disc-like portion 11 a predetermined expanded diameter and a shaft 16 in the center of the disc-like portion. A “shaft” refers to a center portion of a closure member 10, and it may include a rod, tube, a series of wires, which are braided or loose, or otherwise brought together. The metal fabric from which the basket is formed may comprise a plurality of wire strands that may be woven or braided into a tubular configuration and then heat set in a mold in a manner described in U.S. Pat. No. 6,123,715, the contents of which are hereby incorporated by reference.

As illustrated in FIG. 5B, one characteristic of a basket is that when the basket is in its expanded configuration, once deployed in a vessel, it becomes flat or the disc-like and forms about 90 degrees angle with the shaft in the center of the basket. Once placed in the puncture site, this angle assures that when occlusive material is injected through a sideport of a catheter and placed outside the vessel but adjacent to the puncture site, the occlusive material will not enter the vessel through the puncture site.

A collapsible basket may also comprise a plurality of loops attached to a shaft, the loops interleaved and formed into an atraumatic periphery of the basket.

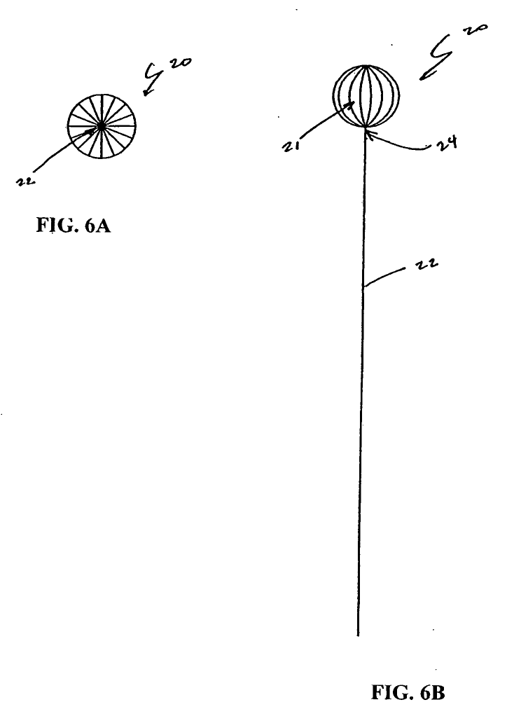

A collapsible basket may be in a shape of sphere, as shown in FIGS. 6A and 6B. FIG. 6B illustrates a closure member 20, which comprises a basket 21 made of plurality of loops of wire, interweaved or interlaced to form the basket 21. The loops are joined into a cannula or joining portion 24 of a shaft which may extend to a handle (not shown) for use by a surgeon or technician using the closure member. Basket 21 includes a periphery with a flex point 26 for easier collapsing of the basket.

A basket used in this invention may be made, for example, from a metal fabric by deforming a metal fabric to generally conform to a molding surface of a molding element and heat treating the fabric to substantially set the fabric in its deformed state.

When forming these baskets from a resilient metal fabric a plurality of resilient strands may be provided, with the wires being formed by braiding to create a resilient material which may be heat treated to substantially set a desired shape. This braided fabric may then be deformed to generally conform to a molding surface of a molding element and the braided fabric may be heat treated in contact with the surface of the molding element at an elevated temperature. The time and temperature of the heat treatment may be selected to substantially set the braided fabric in its deformed state. After the heat treatment, the fabric may be removed from contact with the molding element and will substantially retain its shape in the deformed state. The braided fabric so treated defines an expanded state of a medical device which may be deployed through a catheter into a channel in a patient's body.

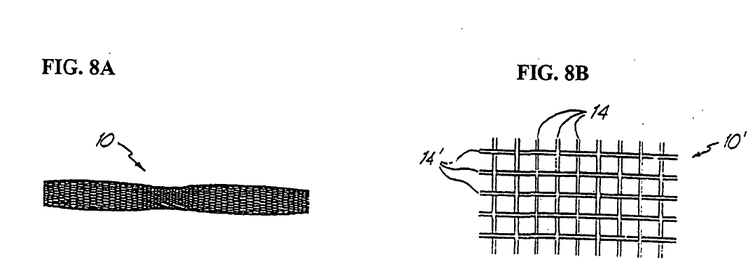

FIGS. 8A and 8B illustrate two examples of metal fabrics which are suitable for use to form a basket of the closure member. In the fabric of FIG. 8A, the metal strands define two sets of essentially parallel generally helical strands, with the strands of one set having a “hand”, i.e. a direction of rotation, opposite that of the other set. This defines a generally tubular fabric, known in the fabric industry as a tubular braid. Such tubular braids are well known in the fabric arts and find some applications in the medical device field as tubular fabrics, such as in reinforcing the wall of a guiding or diagnostic catheter. As such braids are well known, they need not be discussed at length here.

The pitch of the wire strands (i.e., the angle defined between the turns of the wire and the axis of the braid) and the pick of the fabric (i.e., the number of turns per unit length) may be adjusted as desired for a particular application. For example, if the medical device to be formed is to be used to occlude the puncture site in which it is placed according to this invention, the pitch and pick of the fabric will tend to be high.

For example, in using a tubular braid such as that shown in FIG. 8A, a tubular braid of about 4 mm in diameter with a pitch of about 50° and a pick of about 74 (per linear inch) would seem suitable for fabricating devices used to form baskets suitable for occluding puncture site on the order of about 2 mm to about 4 mm in inner diameter.

FIG. 8B illustrates another type of fabric which is suitable for use in the method of the invention. This fabric is a more conventional fabric and may take the form of a flat woven sheet, knitted sheet or the like. In the woven fabric shown in FIG. 8B, there may be also two sets 14 and 14′ of generally parallel strands, with one set of strands being oriented at an angle, e.g., generally perpendicular (having a pick of about 90°), with respect to the other set. As noted above, the pitch and pick of this fabric (or, in the case of a knit fabric, the pick and the pattern of the knit, e.g., Jersey or double knits) may be selected to optimize the desired properties of the final medical device.

The wires of the basket portion of the closure member are preferably made of a superelastic or shape memory alloy, such as Nitinol, a nickel-titanium alloy. The wires may also be made from other shape memory metals, such as alloys of Cu—Zn—Al or Cu—Al—Ni. In order to keep the size of the basket and the diameter of the sheath narrow, very thin wires are preferred, such as wires having a diameter of about 0.0025 inches (about 0.063 mm). Round wires are preferred, but wires of any shape may be used, including rectangular wire, square wire, wedge or “pie-shaped” wire, flat wire and triangular wire. Each “wire” in reality may comprise two or more wires twisted together for greater stiffness and control of the device.

As is well known in the art, the wires may be formed into a desired shape and heat treated or “trained” into that shape by heating to a certain temperature for a certain length of time. Typically, temperatures in the range of 500-540° C. and times from 1-5 minutes are used. Other temperatures and times may also be used. Shape-memory or superelastic materials are heat treated or annealed from a weak (martinsite) structure to a strong (austenite) structure. The alloys are weak and deformable in the martinsitic state, which is thus useful for forming the basket and the loops. After transformation to the strong or austenitic state, they exhibit a superelastic property so long as the material remains above a transformation temperature, at which temperature it will revert to the martinsitic state. The transformation temperature may be desirably a low temperature, well below the temperature of a human body, and preferably below room temperature, which is about 20-25° C. The transformation temperature of the wires and the basket may thus be selected to be below the operating temperature of the basket, thus keeping the basket in a superelastic state. In this state, the wires advantageously return to their original, unstressed shape when deforming stresses are removed. The superelastic wire alloy also increasingly resists deformation as the stress load is increased. Thus, when a superelastic basket is collapsed and placed into the sheath, the loops forming the basket are placed into a state of stress. When the loops are deployed, the stresses are removed, and the loops return to the desired shape of a basket.

The baskets may be formed by shaping the wires and loops into the desired shape at room temperature or below, preferably with a cold mandrel, and then annealing the properly-shaped basket at the proper annealing temperature for a time sufficient for the transformation to a superelastic state. In one example, a basket may be formed from 0.11 mm diameter (about 0.0043 inches) Ni—Ti Nitinol wire and is annealed at 990° F. (about 530° C.) for about 10 minutes. The time and temperature for annealing may vary with the alloy selected and with the diameter (thickness) of the wire. The loops themselves, not merely the annealing oven, must remain at the desired temperature for the proper length of time for the annealing or heat-treatment to be complete. Proper annealing is very important for the wires and the loops to remain kink-free during deployment and operation of the basket. If kinks form for any reason, it may be difficult to deploy (expand) or retract the basket.

The basket may be preferably formed before the annealing operation, as discussed above, including all wires or loops in the asymmetric basket. Because of the non-symmetrical shape of the basket, it may be possible that it may require more force or more built-in stress in the wires to reliably emerge from the sheath in the desired shape. Therefore, the annealing or heat-treating operation is even more important than normal in building stresses into the wires and the basket.

The basket and the wires may be “trained” in the shape of the deployed basket. They may also be joined to a joining portion at the distal end of a control rod. Control rod may be a solid Nitinol rod or tube, or may be a stainless steel shaft or tube. Nitinol is preferred. The control rod may instead be a number of stranded or non-stranded wires, depending on the degree of flexibility desired. Joining portion may simply be a separate hollow cannula or a hollowed-out portion at the distal end of the control rod or control tube. The wires from the basket may be trimmed and joined to the end of the control rod by one or more of several means.

For example, the ends of the wire strands forming the metal basket may be attached to one another to prevent the fabric from unraveling as schematically illustrated in FIG. 7B by a clamp 32. The clamp 32 may also serve to connect the device 30 to a delivery system (not shown). In the embodiment shown, the clamp 30 is generally cylindrical in shape and has a recess for receiving the ends of the wires to substantially prevent the wires from moving relative to one another, and a threaded outer surface. The threaded outer surface may be adapted to be received within a cylindrical recess (not shown) on a distal end of a delivery device and to engage the threaded inner surface of the delivery device's recess.

The ends of the wire may also be attached to one another and/or the shaft by other methods, such as by welding, soldering, brazing, use of a biocompatible cementitious material or in any other suitable fashion.

A medically-acceptable adhesive may also be used to secure or join the wires to the shaft. Loctite® 4011 cyanoacrylate may be used for this application. The wires from the basket may themselves extend to a control handle, rather than using a separate connector and shaft. In one embodiment, the closure member comprises loops or wires with ends connected to the shaft. A separate cannula may be used to connect the wires or loops to the shaft. The cannula may be joined to the shaft, preferably by soldering, although other techniques, such as welding or brazing may also be used. If soldering is used, the shaft may be first etched, preferably with acid, followed by neutralizing and drying. Flux may then be applied to both the shaft and the cannula, the two may be soldered together, and excess solder may be removed. Afterwards, the parts should be neutralized, dried and cleaned.

FIGS. 7A and 7B illustrate yet another example of a closure member, which may be well suited for use in the medical closure system of this invention. This closure member 30 has a generally umbrella-shaped body 31 and a shaft 32. This type of a closure member is a modification of devices, which were previously described in the art to occlude defects known in the art as central shunts or patent ductus arteriosus (PDA). See, for example, U.S. Pat. No. 5,725,552, which is incorporated herein in its entirety.

The umbrella-shaped body 31 and the shaft 33 can be deployed within a blood vessel or a body cavity and are adapted to be positioned within the wall of the blood vessel or body cabity. The sizes of the body 31 can be varied as desired for differently sized puncture sites. In one example, the diameter of the body may be to smaller than the diameter of the blood vessel or body cavity but of diameter to fully occlude the puncture. For example, the body may have a diameter of about 5 mm to about 100 mm.

A closure member may also include a Malecot assembly. Malecot assembly and methods of deploying Malecot assembly were previously described in U.S. Pub. No. 2004/0225322 A1, disclosure of which is incorporated herein in its entirety.

The Malecot assembly may be a suitable means for defining area for delivery of the occlusive material and preventing the occlusive material from entering the blood vessel or body cavity at the puncture. Malecot assemblies are known in the medical technology art and are commonly used to provide drainage egress from a body cavity. Malecot assemblies have not, however, been used as closure members as described in this specification. U.S. Pat. No. 2,649,092 provides a description of a Malecot assembly, and is incorporated by reference into this disclosure in its entirety for the purpose of describing a Malecot assembly.

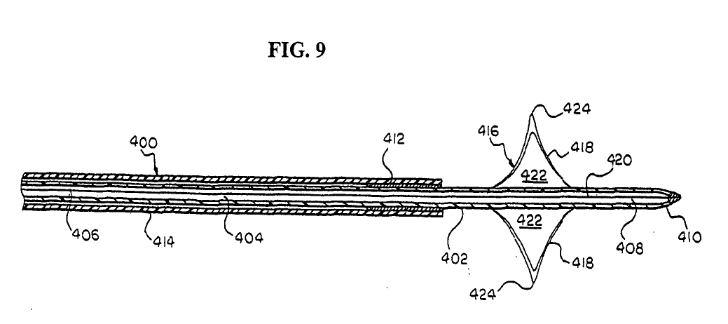

Briefly, referring to FIG. 9, the Malecot assembly 416 comprises two or more strip-like sections 418 of material that are formed by slits in the material of the elongate member 402. An elongate activator 420 may be attached to the distal end 408 of the elongate member and extends through the elongate member 402 to the proximal end 406. To activate the Malecot assembly 416, a user may pull the elongate activator 420 toward the proximal end 406 of the elongate member 402. This action may enlarge the slits in the elongate member 402 to create open spaces 422 and force the strip-like sections 418 to fold and extend radially outward. The radially-outward extending strip-like sections 418 of material space the elongate member 402 from a surface contacting a fold 424 in the sections 418, such as an interior wall surface of a body vessel. To deactivate the Malecot assembly and substantially return the strip-like sections 418 to their original position, the user may release the elongate activator 420. A pusher (not illustrated) may be advanced through the lumen of the elongate member 402 to push on the distal end 408 to facilitate deactivation of the Malecot assembly 416.

In addition to collapsible baskets and Malecot assembly, other suitable devices which may be capable of defining area for delivery of the occlusive material a puncture site and sealing a puncture, may also be used. Exemplary devices, such as an expandable diaphragm previously described in WO 03/049622, disclosure of which is incorporated herein in its entirety, may also be used. Expandable diaphragm may preferably comprise a polymer membrane supported by superelastic hoop of nickel-titanium wire, for example.

It is therefore intended that the foregoing detailed description be regarded as illustrative rather than limiting, and that it be understood that it is the following claims, including all equivalents, that are intended to define the spirit and scope of this invention.

1. A method for sealing a puncture through a wall of a blood vessel or wall of a body cavity, comprising the steps of deploying a closure member in a first compacted configuration through a delivery member into the blood vessel or body cavity though the puncture, wherein the closure member radially expands to assume a second expanded configuration following the deployment;
positioning the closure member on an inner surface of the blood vessel or body cavity at the puncture;
delivering a reconstituted or naturally-derived collagenous material and a hemostatic material through the delivery member to an outer surface of the blood vessel or body cavity at the puncture;
wherein the closure member defines an area for delivering of the reconstituted or naturally-derived collagenous material and the hemostatic material at the puncture.

2. The method of claim 1, further comprising retracting the closure member through the delivery member.

3. The method of claim 1, wherein the reconstituted or naturally-derived collagenous material and the hemostatic material are delivered via at least one sideport positioned on a distal end of the delivery member.

4. The method of claim 1, wherein the puncture is a vascular puncture made during a vascular, endoscopic, or orthopaedic surgical procedures.

5. A method for sealing a puncture through a wall of a blood vessel or wall of a body cavity, comprising the steps of
deploying a closure member in a first compacted configuration through a delivery member into the blood vessel or body cavity though the puncture, wherein the closure member radially expands to assume a second expanded configuration following the deployment;
positioning the closure member on an inner surface of the blood vessel or body cavity at the puncture;
delivering a composition comprising a reconstituted or naturally-derived collagenous material and a hemostatic material through the delivery member to an outer surface of the blood vessel or body cavity at the puncture;
wherein the closure member defines an area for delivering of the reconstituted or naturally-derived collagenous material and the hemostatic material at the puncture.

6. The method of claim 5, further comprising retracting the closure member through the delivery member.

7. The method of claim 5, wherein the reconstituted or naturally-derived collagenous material and the hemostatic material is delivered via at least one sideport positioned on a distal end of the delivery member.

8. The method of claim 5, wherein the puncture is a vascular puncture made during a vascular, endoscopic, or orthopaedic surgical procedures.

9. A medical closure system for sealing a puncture through a wall of a blood vessel or wall of a body cavity, comprising:
a closure member which can be in a first collapsed configuration or in a second expanded configuration;
a reconstituted or naturally-derived collagenous material; and
a hemostatic material.

10. The system of claim 9, further comprising a delivery member.

11. The system of claim 10, wherein the delivery member comprises at least one sideport positioned at a distal end of the delivery member.

12. The system of claim 9, wherein the reconstituted or naturally-derived collagenous material comprises an ECM material.

13. The system of claim 12, wherein the ECM is in an injectable form.

14. The system of claim 12, wherein the ECM is impregnated with the hemostatic material.

15. The system of claim 9, wherein the hemostatic material is selected from the group consisting of HEMOS, Fibrin adhesive material, Epsilon-Aminocapronic Acid, Chitosan, poly-N-acetylglucosamine, Microporous polysaccharide hemosphere, QR powder, and hemostatic lipid.

16. The system of claim 9, wherein the hemostatic material is HEMOS.

17. The system of claim 9, wherein the closure member comprises a collapsible basket.

18. The system of claim 9, wherein the closure member comprises a Malecot assembly.

19. The system of claim 9, wherein the closure member is an expandable diaphragm.

20. A medical closure system for sealing a puncture through a wall of a blood vessel or in the wall of a body cavity comprising:
a closure member which can be in a first collapsed configuration or in a second expanded configuration;
a composition comprising a reconstituted or naturally-derived collagenous material and a hemostatic material.
